(19) United States
(12) Patent Application Publication
(10) Pub. No.: US 2022/0017436 A1
(43) Pub. Date: Jan. 20, 2022

(54) SEPARATION OF OLEFIN COMPONENTS FROM A MIXTURE OF BUTANES AND BUTENES USING DISTILLATION AND ADSORBENTS

(71) Applicant: SABIC GLOBAL TECHNOLOGIES B.V., Bergen op Zoom (NL)

(72) Inventors: Mohammed ALDOSSARY, Riyadh (SA); Mohammed Nadim SHAIK, Riyadh (SA); Khalid A. AL-MAJNOUNI, Riyadh (SA); Ahmed ALZENAIDI, Riyadh (SA); Faizur RAHMAN, Dhahran (SA); Arudra PALANI, Dhahran (SA); Sachio ASAOKA, Yokohama (JP); Mohammad Ahmed AL-YAMI, Dhahran (SA); Uwais BADURUTHAMAL, Dhahran (SA)

(73) Assignee: SABIC Global Technologies B.V., Bergen op Zoom (NL)

(21) Appl. No.: 17/414,821

(22) PCT Filed: Dec. 17, 2019
(86) PCT No.: PCT/IB2019/060925

§ 371 Date: Jun. 16, 2021

Related U.S. Application Data
(60) Provisional application No. 62/781,202, filed on Dec. 18, 2018.

(51) Int. Cl. C07C 7/04; C07C 7/13

(52) U.S. Cl. CPC C07C 7/04; C07C 7/13

(57) ABSTRACT

Systems and methods for separating a mixture of olefinic and paraffinic C₄ components are disclosed. The systems and methods include adsorptive equipment and adsorptive processes for separating components from the mixture.

### CROSS REFERENCE TO RELATED APPLICATIONS

This application claims priority to and the benefit of U.S. Provisional Application No. 62/781,202, filed Dec. 18, 2018, the contents of which is incorporated into the present application in its entirety.

### FIELD OF INVENTION

The present invention generally relates to the separation of multicomponent hydrocarbon streams. More specifically, the present invention relates to the separation of a mixture that includes butanes and butenes using distillation and adsorption processes.

### BACKGROUND OF THE INVENTION

Steam cracking units and fluid catalytic crackers (FCC) of olefin plants produce mixtures of C₄ fractions. Typically, butadiene is extracted from a mixture of C₄ fractions and after extracting the butadiene from this mixture, a resulting mixed C₄ stream of n-butane, isobutane (i-butane), isobutene (i-butene) along with n-butenes, typically remains. Conventionally, the 1-butene and i-butene in the mixed C₄ stream are separated from each other using a process that synthesizes methyl tertiary butyl ether (MTBE). In this process, the mixed C₄ stream is processed in a reactor where i-butene is converted to MTBE using methanol as a reactant. After reaction, the i-butene depleted mixed C₄ stream is separated from MTBE and methanol using reactive distillation and conventional distillation processes. The distillation processes recover the butenes (1-butene, cis-2-butene (c-butene), and trans-2-butene (t-butene), whilst the C₄ paraffins are recycled, for example, to the catalytic cracker.

### BRIEF SUMMARY OF THE INVENTION

A method has been discovered for separating a mixture of olefinic and paraffinic C₄ fractions obtained from steam cracking or fluid catalytic crackers into its components. The method can involve the separation of isobutene and 1-butene from a mixed stream comprising olefinic and paraffinic C₄s.

Embodiments of the invention include, a method of recovering olefins from a C₄ hydrocarbon mixture. The method comprises fractionating the C₄ hydrocarbon mixture in a first separation section to form (1) a first olefin stream comprising primarily 1-butene and isobutene collectively and (2) a first byproduct stream that comprises primarily cis-2-butene and trans-2-butene collectively. The method further comprises separating the first olefin stream to form an isobutene stream comprising primarily isobutene and a 1-butene stream comprising primarily 1-butene via a second separation section. The second separation section is adapted to separate hydrocarbon streams by adsorption.

Embodiments of the invention include, a method of recovering olefins from a C₄ hydrocarbon mixture. The method comprises separating the C₄ hydrocarbon mixture in an adsorber section to form (1) a first olefin stream that comprises primarily 1-butene and isobutene collectively and (2) a first byproduct stream that comprises primarily cis-2-butene, trans-2-butene, n-butane, and isobutane collectively. The method further comprises separating the first olefin stream, via molecular sieves, into an isobutene stream comprising primarily isobutene and a 1-butene stream comprising primarily 1-butene.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “primarily,” as that term is used in the specification and/or claims, means greater than any of 50 wt. %, 50 mol. %, or 50 vol. %. For example, “primarily” may include 50.1 wt. % to 100 wt. % and all values and ranges there between, 50.1 mol. % to 100 mol. % and all values and ranges there between, 50.1 vol. % to 100 vol. % and all values and ranges there between.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unnamed elements or method steps.

The process of the present invention can “comprise,” “consist essentially of,” or “consist of” particular ingredients, components, compositions, etc., disclosed throughout the specification.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a system for recovering olefins from a C₄ hydrocarbon mixture, according to embodiments of the invention;

FIG. 2 shows a method for recovering olefins from a C₄ hydrocarbon mixture, according to embodiments of the invention;

FIG. 3 shows a system for recovering olefins from a C₄ hydrocarbon mixture, according to embodiments of the invention;

FIG. 4 shows a method for recovering olefins from a C₄ hydrocarbon mixture, according to embodiments of the invention;

FIG. 5 shows a system for recovering olefins from a C₄ hydrocarbon mixture, according to embodiments of the invention; and

FIG. 6 shows a method for recovering olefins from a C₄ hydrocarbon mixture, according to embodiments of the invention.

### DETAILED DESCRIPTION OF THE INVENTION

A method has been discovered for separating, into its components, a mixture of olefinic and paraffinic C₄ fractions obtained from steam cracking or fluid catalytic crackers. The method can involve the separation of isobutene and 1-butene from a mixed stream comprising olefinic and paraffinic C₄s.

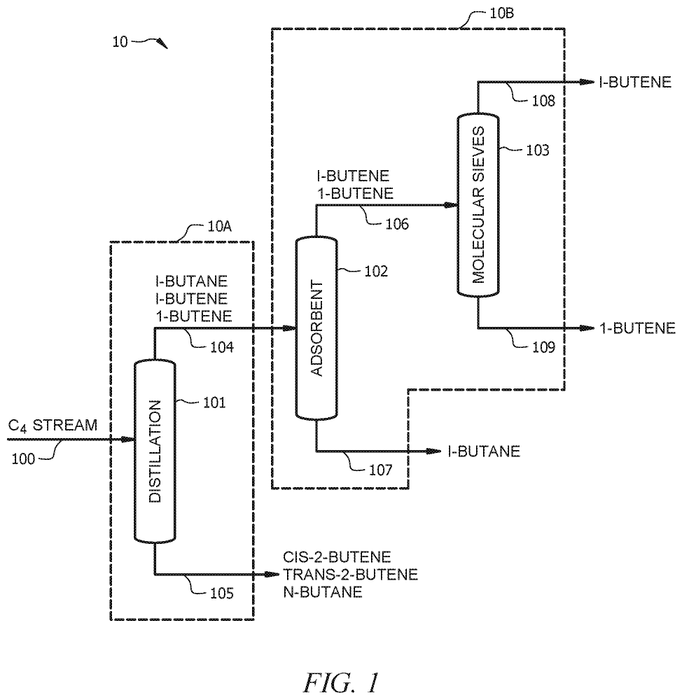

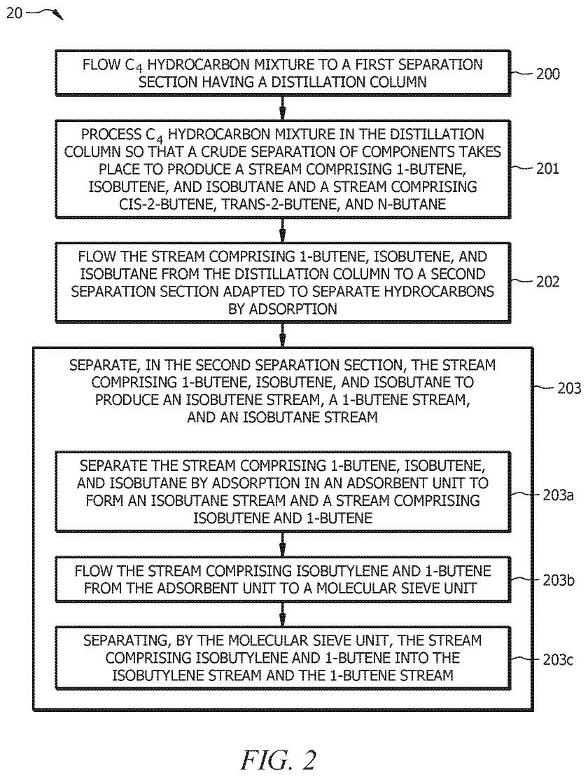

FIG. 1 shows system 10 for recovering olefins from a C₄ hydrocarbon mixture, according to embodiments of the invention. System 10 involves the integration of distillation equipment and adsorption equipment for use in a method of recovering olefins from a C₄ hydrocarbon mixture. FIG. 2 shows method 20 for recovering olefins from a C₄ hydrocarbon mixture, according to embodiments of the invention. Method 20 may be implemented using system 10.

FIG. 1 shows the integration of distillation column 101, adsorption unit 102, and molecular sieve unit 103. As shown, first separation section 10A may comprise distillation column 101 and second separation section 10B may comprise one or more adsorber unit(s) such as adsorption unit 102 and molecular sieve unit 103. According to embodiments of the invention, method 20, as implemented by system 10, begins at block 200, which involves flowing C₄ hydrocarbon mixture 100 (from, for example, a steam cracker, a catalytic cracker, a catalytic dehydrogenation unit, or combinations thereof) to first separation section 10A. In embodiments of the invention, effluent from the steam cracker, the catalytic cracker, the catalytic dehydrogenation unit, or combinations thereof, is subjected to a process that removes butadiene, which results in the formation of C₄ hydrocarbon mixture 100, which comprises isobutane, isobutene, 1-butene, cis-2-butene, trans-2-butene, and n-butane. In embodiments of the invention, C₄ hydrocarbon mixture 100 comprises 1.0 mol. % to 2.0 mol. % isobutane, 16.0 mol. % to 26.0 mol. % isobutene, 35 mol. % to 53 mol. % 1-butene, 5.0 mol. % to 9.0 mol. % cis-2-butene, 12.0 mol. % to 22.0 mol. % trans-2-butene, and 4.0 mol. % to 10.0 mol. % n-butane.

At block 201, distillation column 101 processes C₄ hydrocarbon mixture 100 such that a crude separation of components takes place. Block 201 may include distillation column 101 distilling C₄ hydrocarbon mixture 100 to produce a distillate, namely first olefin stream 104, and a bottom stream, namely first byproduct stream 105. According to embodiments of the invention, first olefin stream 104 comprises primarily (greater than 50 wt. %) 1-butene and isobutene collectively and first byproduct stream 105 comprises primarily cis-2-butene and trans-2-butene collectively. In embodiments of the invention, first olefin stream 104 comprises 0.1 mol. % to 4.0 mol. % isobutane, 20 mol. % to 40 mol. % isobutene, and 60 mol. % to 70 mol. % 1-butene, and 0.1 mol. % to 1 mol. % cis-2-butene, trans-2-butene, and n-butane, collectively. In embodiments of the invention, first byproduct stream 105 comprises 18 mol. % to 26 mol. % cis-2-butene, 50 mol. % to 60 mol. % trans-2-butene, 18 mol. % to 26 mol. % n-butane, and 0.5 mol. % to 2.0 mol. % 1-butene, isobutene, and isobutane, collectively. The distillation conditions for distillation column 101 may include a temperature in a range of 20° C. to 100° C. and a pressure in a range of 1 bar to 20 bars.

Method 20 continues at block 202, which involves flowing first olefin stream 104 from distillation column 101 (first separation section 10A) to second separation section 10B, where first olefin stream 104 is separated, at block 203, to produce isobutene stream 108, which comprises primarily isobutene, 1-butene stream 109, which comprises primarily 1-butene, and isobutane stream 107, which comprises primarily isobutane. Second separation section 10B is adapted to separate hydrocarbon streams at least by adsorption, for example, by the use of adsorption unit 102, and molecular sieve unit 103.

According to FIG. 1 and FIG. 2, block 203 may be carried out by block 203a to block 203c. Block 203a may include adsorption unit 102, in second separation section 10B, processing first olefin stream 104 (which includes 1-butene, isobutene and isobutane) such that isobutane stream 107 (comprising primarily isobutane) is separated from second olefin stream 106 (a mixture comprising primarily isobutene and 1-butene collectively). This separation is achieved using an adsorption process in adsorption unit 102.

According to embodiments of the invention, block 203 further includes, at block 203b, flowing second olefin stream 106 from adsorption unit 102 to molecular sieve unit 103. At block 203c, method 20 may further include separating, by molecular sieve unit 103, second olefin stream 106 into isobutene stream 108 (comprising primarily isobutene) and 1-butene stream 109 (comprising primarily 1-butene). In embodiments of the invention, isobutene stream 108 comprises 90 wt. % to 99.9 wt. % isobutene, and 0.1 wt. % to 10 wt. % 1-butene and others. In embodiments of the invention, 1-butene stream 109 comprises 90 wt. % to 99.9 wt. % 1-butene, and 0.1 wt. % to 10 wt. % isobutene and others.

The adsorption process that occurs in adsorption unit 102 and the molecular sieve process that occurs in molecular sieve unit 103, both achieve separation based on the principle of adsorption. According to embodiments of the invention, a difference between the adsorption process that occurs in adsorption unit 102 and the molecular sieve process that occurs in molecular sieve unit 103 is the type of material used to achieve the separation. The principle of operation that involves adsorption that is described herein is thus relevant to both the adsorption process that occurs in adsorption unit 102 and the molecular sieve process that occurs in molecular sieve unit 103, which are both transient processes. Adsorption unit 102 and molecular sieve unit 103 can include multiple vessels (e.g., two or three). Each of adsorption unit 102 and molecular sieve unit 103 can contain one or more beds of adsorbent material. In embodiments of the invention, the adsorbent of adsorption unit 102 includes zeolite (silica and alumina), metal (either copper, potassium, sodium), or combinations thereof. In embodiments of the invention, the molecular sieves of molecular sieve unit 103 include 5A, or 13X, or Y-zeolite or modified 13X or Y-zeolite, or silicalite or high silica ZSM-5, or combinations thereof. The isobutene adsorbed in molecular sieve unit 103 can be desorbed either by pressure swing adsorption (PSA)/desorption process or temperature swing adsorption (TSA)/desorption process. The adsorbed species is desorbed from the molecular sieve by making changes in the pressure or temperature. If pressure swing is used, then in order to desorb the adsorbed species, a reduction in pressure is required. If a temperature swing process is used, then increasing the temperature results in desorption of the adsorbed species.

As the fluid being separated moves through the bed of adsorption unit 102 and the bed of molecular sieve unit 103, it comes into contact with the adsorbent at the entrance of the bed and certain molecules are adsorbed. The molecules that are adsorbed are referred to as adsorbate. At the onset of the adsorption process, the region at the entrance of the bed is referred to as the active zone or mass transfer zone (MTZ). As time progresses, the adsorbent material at the bed entrance becomes saturated (meaning that the rate of adsorption equals the rate of desorption) and the MTZ moves further down the length of the bed. The region in the bed which is saturated is referred to as the equilibrium zone (EZ). After a sufficient amount of time has passed, the entire bed is at equilibrium and breakthrough of adsorbate occurs. At this point the bed would not be effective in separating the fluid that needs separation.

Thus, for a continuous uninterrupted process, in carrying out method 20, multiple vessels (each comprising a bed of adsorbent material) are employed and these vessels are operated in a staggered manner. For example, one vessel with a bed of adsorption unit 102 and one vessel with a bed of molecular sieve unit 103 is in operation or adsorption mode, whilst another vessel with a bed of adsorption unit 102 and another vessel with a bed of molecular sieve unit 103 is in regeneration mode and optionally a third vessel with a bed of adsorption unit 102 and a third vessel with a bed of molecular sieve unit 103 is in standby mode.

According to embodiments of the invention, in method 20, when a particular bed of adsorption unit 102 becomes saturated, the fluid flow can be diverted to a bed of adsorption unit 102 that is in standby mode. Similarly, when a particular bed of molecular sieve unit 103 becomes saturated, the fluid flow can be diverted to a bed of molecular sieve unit 103 that is in standby mode. The respective saturated bed is then regenerated by desorbing the adsorbate.

A number of techniques are available and can be implemented to regenerate a saturated bed as needed in method 20. These include a reduction in pressure of the saturated bed (pressure swing), increasing the temperature of the saturated bed (temperature swing) or passing an inert gas or solvent through the saturated bed, which strips the adsorbate from the adsorbent material. In embodiments of the invention, second separation section 10B comprises a selection from the list consisting of: a thermal swing adsorber, a pressure swing adsorber, and combinations thereof.

In embodiments of the invention, adsorption unit 102 comprises a thermal swing adsorber that includes adsorbent that comprises 5A or 13X or Y-zeolite or modified 13X or Y-zeolite or silicalite or high silica ZSM-5, or combinations thereof. In embodiments of the invention, adsorption unit 102 comprises a pressure swing adsorber that includes absorbent that comprises 5A, or 13X, or Y-zeolite or modified 13X or Y-zeolite or silicalite or high silica ZSM-5 or combinations thereof. In embodiments of the invention, method 20 includes, at block 203, desorbing isobutene from the molecular sieves of molecular sieve unit 103.

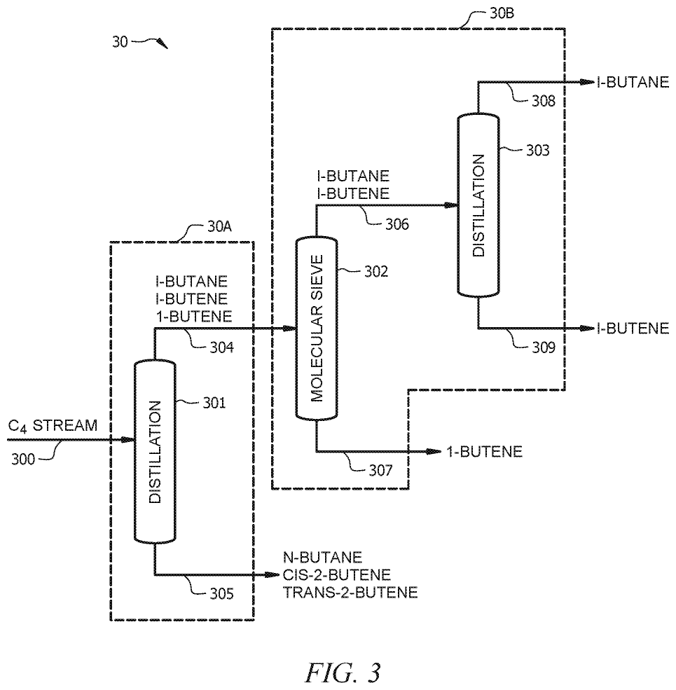

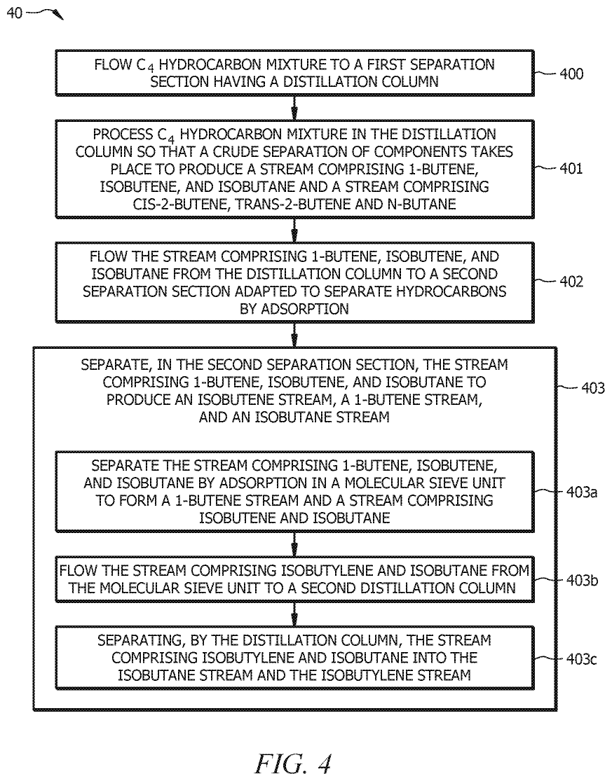

FIG. 3 shows system 30 for recovering olefins from a C₄ hydrocarbon mixture, according to embodiments of the invention. System 30 involves the integration of distillation equipment and adsorption equipment for use in a method of separating olefins from a C₄ hydrocarbon mixture. FIG. 4 shows method 40 for recovering olefins from a C₄ hydrocarbon mixture, according to embodiments of the invention. Method 40 may be implemented using system 30.

FIG. 3 shows integration of distillation column 301, molecular sieve unit 302, and distillation column 303. As shown, first separation section 30A may comprises distillation column 301 and second separation section 30B may comprise one or more separation unit(s) such as molecular sieve unit 302 and distillation column 303. According to embodiments of the invention, method 40, as implemented by system 30, begins at block 400, which involves flowing C₄ hydrocarbon mixture 300 (from, for example, a steam cracker, a catalytic cracker, a catalytic dehydrogenation unit, or combinations thereof) to first separation section 30A. In embodiments of the invention, effluent from the steam cracker, the catalytic cracker, the catalytic dehydrogenation unit, or combinations thereof, is subjected to a process that removes butadiene, which results in the formation of C₄ hydrocarbon mixture 300, which comprises isobutane, isobutene, 1-butene, cis-2-butene, trans-2-butene, and n-butane. In embodiments of the invention, C₄ hydrocarbon mixture 300 comprises 1.0 mol. % to 2.0 mol. % isobutane, 16.0 mol. % to 26.0 mol. % isobutene, 35 mol. % to 53 mol. % 1-butene, 5.0 mol. % to 9.0 mol. % cis-2-butene, 12.0 mol. %to 22.0 mol. % trans-2-butene, and 4.0 mol. % to 10.0 mol. % n-butane.

At block 401, distillation column 301 processes C₄ hydrocarbon mixture 300 such that a crude separation of components takes place. Block 401 may include distillation column 301 distilling C₄ hydrocarbon mixture 300 to produce a distillate, namely first olefin stream 304, and a bottom stream, namely first byproduct stream 305. According to embodiments of the invention, first olefin stream 304 comprises primarily (greater than 50 wt. %) 1-butene and isobutene collectively and first byproduct stream 305 comprises primarily cis-2-butene and trans-2-butene collectively. In embodiments of the invention, first olefin stream 304 comprises 0.1 mol. % to 4.0 mol. % isobutane, 20 mol. % to 40 mol. % isobutene, and 60 mol. % to 70 mol. % 1-butene, and 0.1 mol. % to 1 mol. % cis-2-butene, trans-2-butene, and n-butane, collectively. In embodiments of the invention, first byproduct stream 305 comprises 18 mol. % to 26 mol. % cis-2-butene, 50 mol. % to 60 mol. % trans-2-butene, 18 mol. % to 26 mol. % n-butane, and 0.5 mol. % to 2.0 mol. % 1-butene, isobutene, and isobutane, collectively. The distillation conditions for distillation column 301 may include temperature in a range of 20° C. to 100° C. and pressure in a range of 1 bar to 20 bars.

Method 40 continues at block 402, which involves flowing first olefin stream 304 from distillation column 301 (first separation section 30A) to second separation section 30B, where first olefin stream 304 is separated, at block 403, to produce isobutane stream 308, which comprises primarily isobutane, isobutene stream 309, which comprises primarily isobutene, and 1-butene stream 307, which comprises primarily 1-butene. Second separation section 30B may be adapted to separate hydrocarbon streams at least by adsorption; for example, by the use of molecular sieve unit 302. Additionally, distillation column 303 may be used for further separating as shown.

According to FIG. 3 and FIG. 4, block 403 may include block 403a, where molecular sieve unit 302, in second separation section 30B, processes first olefin stream 304 (which includes 1-butene, isobutene and isobutane) such that 1-butene stream 307 (comprising primarily 1-butene) is separated from second olefin stream 306 (a mixture comprising primarily isobutane and isobutene collectively). This separation is achieved using an adsorbent process in molecular sieve unit 302.

According to embodiments of the invention, block 403 further includes block 403b, which involves flowing second olefin stream 306 from molecular sieve unit 302 to distillation column 303. At block 403c, method 40 may further include separating, by distillation column 303, second olefin stream 306 into isobutane stream 308 (comprising primarily isobutane) and isobutene stream 309 (comprising primarily isobutene). In embodiments of the invention, isobutane stream 308 comprises 90 wt. % to 99.9 wt. % isobutane, and 0.1 wt. % to 10 wt. % isobutene and others. In embodiments of the invention, isobutene stream 309 comprises 90 wt. % to 99.9 wt. % 1-butene, and 0.1 wt. % to 10 wt. % isobutene and others. In embodiments of the invention described herein, the rate of recovering isobutene from the C₄ hydrocarbon mixture is at least 60 wt. % or preferably more than 80 wt. % or more preferably greater than 90 wt. % and rate of recovering 1-butene from the C₄ hydrocarbon mixture is 60 wt. % or preferably more than 80 wt. % or more preferably greater than 90 wt. %. In embodiments of the invention, the purity of the isobutene is at least 90 wt. % or preferably more than 95 wt. % or more preferably greater than 99 wt. %; and the purity of the 1-butene is at least 90 wt. % or preferably more than 95 wt. % or more preferably greater than 99 wt. %.

The mechanisms of the adsorption process that occurs in molecular sieve unit 302 are the same as described above with respect to molecular sieve unit 103

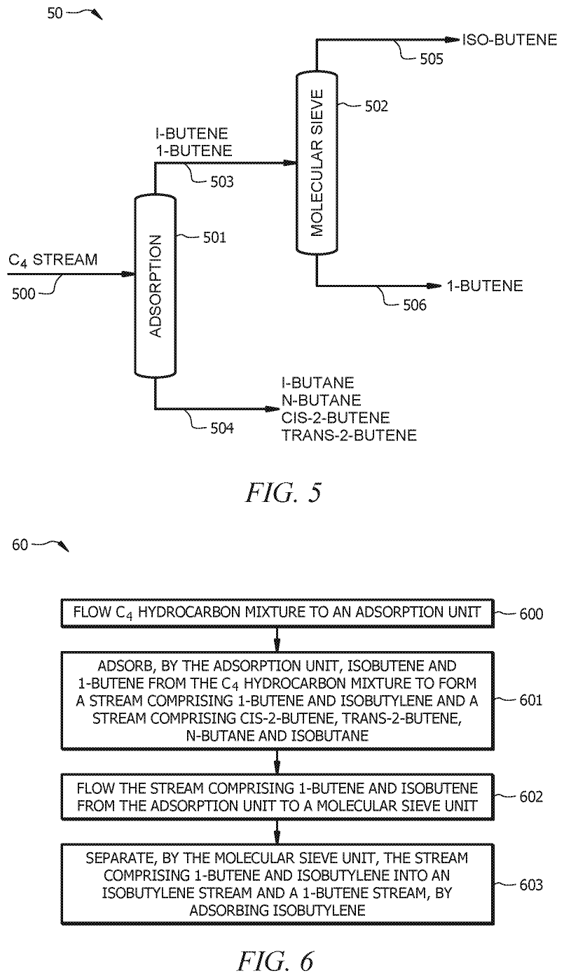

FIG. 5 shows system 50 for separating a C₄ hydrocarbon mixture, according to embodiments of the invention. System 50 comprises adsorption unit 501 and molecular sieve unit 502. FIG. 6 shows a method for recovering olefins from a C₄ hydrocarbon mixture, according to embodiments of the invention. According to embodiments of the invention, system 50 may be used to implement method 60.

According to embodiments of the invention, method 60 may begin at block 600 which involves flowing C₄ hydrocarbon mixture 500 to adsorption unit 501. C₄ hydrocarbon mixture 500 may have a composition as C₄ hydrocarbon mixture 100. At block 601, adsorption unit 501 adsorbs isobutene and 1-butene from C₄ hydrocarbon mixture 500 to form first olefin stream 503, comprising primarily 1-butene and isobutene collectively and first byproduct stream 504, comprising primarily cis-2-butene, trans-2-butene, n-butane, and isobutane collectively. In embodiments of the invention, first olefin stream 503 comprises 40 wt. % to 80 wt. % 1-butene, and 20 wt. % to 50 wt. % isobutene and 0.1 wt. % to 40 wt. % of any combination of cis-2-butene, trans-2-butene, n-butane and isobutane.. In embodiments of the invention, first byproduct stream 504, comprises 10 wt. % to 30 wt. % cis-2-butene, 40 wt. % to 60 wt. % trans-2-butene, 10 wt. % to 30 wt. % n-butane, 1 wt. % to 10 wt. % isobutane and 0.1 wt. % to 10 wt. % 1-butene and isobutene. At block 602, method 60 may further include flowing first olefin stream 503 from adsorption unit 501 to molecular sieve unit 502. At block 603, molecular sieve unit 502 separates first olefin stream 503 into isobutene stream 505, comprising primarily isobutene and 1-butene stream 506, comprising primarily 1-butene. The separating at block 603 of first olefin stream 503, according to embodiments of the invention, comprises adsorbing isobutene. In embodiments of the invention, isobutene stream 505 comprises 90 wt. % to 99.9 wt. % isobutene and 0.1 wt. % to 10 wt. % 1 butene. In embodiments of the invention, 1-butene stream 506 comprises 90 wt. % to 99.9 wt. % 1-butene and 0.1 wt. % to 10 wt. % isobutene.

It should be noted that, in embodiments of the invention, isobutene is selectively adsorbed, whilst 1-butene is not adsorbed and passes through the adsorbent bed. In embodiments of the invention, 1-butene is selectively adsorbed, whilst iso-butene is not adsorbed and passes through the adsorbent bed. Either a pressure swing or temperature swing process can be utilized to desorb 1-butene or isobutene. The adsorbed species is desorbed from the molecular sieve by making changes in the pressure or temperature. If pressure swing is used then in order to desorb the adsorbed species, a reduction in pressure is required. If a temperature swing process is used, then increasing the temperature results in desorption of the adsorbed species.

Although embodiments of the present invention have been described with reference to blocks of FIG. 2, FIG. 4, and FIG. 6, it should be appreciated that operation of the present invention is not limited to the particular blocks and/or the particular order of the blocks illustrated in FIG. 2, FIG. 4, and FIG. 6. Accordingly, embodiments of the invention may provide functionality as described herein using various blocks in a sequence different than that of FIG. 2, FIG. 4, and FIG. 6.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### EXAMPLES

### A Prophetic Example of Separating a C₄ Stream

A prophetic example of separating a C₄ stream, according to embodiments of the invention is described below. The feed composition is provided in Table 1 below.

TABLE 1
Feed composition

| Components | wt % | mol % |
|---|---|---|
| Iso-Butane | 1.39 | 1.35 |
| N-Butane | 7.66 | 7.42 |
| Trans-2-Butene | 17.6 | 17.7 |
| 1-butene | 44.3 | 44.5 |
| Iso-Butene | 21.9 | 21.9 |
| Cis-2-Buteue | 7.12 | 7.15 |

The stream with the composition shown in Table 1 above is processed in a distillation column to yield a distillate and bottoms stream as displayed in Table 2 and Table 3 respectively.

TABLE 2
Distillate composition

| Distillate | mol % |
|---|---|
| 1-Butene | 65.0 |
| Iso-Butene | 32.3 |
| Iso-Butane | 2.00 |
| Cis-2-Butene, Trans-2-Butene, N-Butane | 0.780 |

TABLE 3
Bottoms stream composition

| Bottoms Stream | mol % |
|---|---|
| Cis-2-Butene | 22.1 |
| Trans-2-Butene | 54.4 |
| N-Butane | 22.0 |
| 1-Butene, Iso-Butene, Iso-Butane | 1.52 |

The distillation is performed at a pressure which results in the use of cooling water as a utility for the condenser and low pressure steam as a utility for the reboiler.

The distillate stream is then further processed in the adsorption separation process, where iso-butene and 1-butene are obtained in one stream, whilst iso-butane and small amounts of trans-2-butene, cis-2-butene and n-butane are obtained in another stream. Lastly the iso-butene and 1-butene are separated using a molecular sieve process.

In embodiments of the invention, stream 106 could contain 50 to 70 wt. % 1-butene, 20 to 40 wt. % isobutene and 0.1 to 10 wt. % isobutane. Stream 107 could contain 90 to 99.9 wt. % isobutane and 0.1 to 10 wt. % 1-butene and isobutene.

In the context of the present invention, embodiments 1-20 are described. Embodiment 1 is a method of recovering olefins from a C₄ hydrocarbon mixture. The method includes fractionating the C₄ hydrocarbon mixture in a first separation section to form: (1) a first olefin stream containing primarily 1-butene and isobutene collectively, and (2) a first byproduct stream that contains primarily cis-2-butene and trans-2-butene collectively. The method also includes separating the first olefin stream to form an isobutene stream containing primarily isobutene and a 1-butene stream containing primarily 1-butene via a second separation section, wherein the second separation section is adapted to separate hydrocarbon streams by adsorption. Embodiment 2 is the method of embodiment 1, wherein the first separation section includes a first distillation column. Embodiment 3 is the method of either of embodiments 1 and 2, wherein the first olefin stream further includes isobutane and the first byproduct stream further includes n-butane. Embodiment 4 is the method of any of embodiments 1 to 3, wherein the second separation section includes one or more adsorber unit(s). Embodiment 5 is the method of any of embodiments 1 to 4, wherein the second separation section separates the first olefin stream into: (a) a second olefin stream containing primarily isobutene and 1-butene collectively, and (b) an isobutane stream containing primarily isobutane. Embodiment 6 is the method of embodiment 5, further including separating the second olefin stream into the isobutene stream containing primarily isobutene and the 1-butene stream containing primarily 1-butene. Embodiment 7 is the method of embodiment 6, wherein the separating of the second olefin stream is carried out by a molecular sieve unit. Embodiment 8 is the method of any of embodiments 1 to 4, wherein the second separation section separates the first olefin stream into: (a) a second olefin stream containing primarily isobutene and isobutane collectively, and (b) a 1-butene stream containing primarily 1-butene. Embodiment 9 is the method of embodiment 8, further including separating the second olefin stream into the isobutene stream containing primarily isobutene and an isobutane stream containing primarily isobutane. Embodiment 10 is the method of embodiment 9, wherein the separating of the second olefin stream is carried out by a second distillation column. Embodiment 11 is the method of any of embodiments 1 to 10, wherein the C₄ hydrocarbon mixture is provided to the first separation section from a selection from the list consisting of a steam cracker, a catalytic cracker, a catalytic dehydrogenation unit, and combinations thereof. Embodiment 12 is the method of any of embodiments 1 to 11, wherein the first olefin stream contains: (a) 40 wt. % to 80 wt. % of the 1-butene from the C₄ hydrocarbon mixture, and (b) 20 wt. % to 50 wt. % of the isobutene from the C₄ hydrocarbon mixture. Embodiment 13 is the method of any of embodiments 1 to 12, wherein the first byproduct stream contains 10 wt. % to 30 wt. % of the cis-2-butene from the C₄ hydrocarbon mixture and 40 wt. % to 60 wt. % of the trans-2-butene from the C₄ hydrocarbon mixture. Embodiment 14 is the method of any of embodiments 1 to 13, wherein the second separation section includes a selection from the list consisting of a thermal swing adsorber, a pressure swing adsorber, and combinations thereof. Embodiment 15 is the method of any of embodiments 1 to 14, wherein the second separation section includes a plurality of vessels having adsorbent beds and, during operation, at least one of the vessels is in adsorption mode and at least one of the vessels is in regeneration mode. Embodiment 16 is the method of any of embodiments 1 to 15, wherein rate of recovering isobutene from the C₄ hydrocarbon mixture is at least 60 wt. % or preferably more than 80 wt. %, or more preferably greater than 90 wt. % and rate of recovering 1-butene from the C₄ hydrocarbon mixture is 60 wt. % or preferably more than 80 wt. %, or more preferably greater than 90 wt. %. Embodiment 17 is the method of any of embodiments 1 to 16, wherein the purity of the isobutene is at least 90 wt. % or preferably more than 95 wt. %, or more preferably greater than 99 wt.% and purity of the 1-butene is at least 90 wt. % or preferably more than 95 wt. %, or more preferably greater than 99 wt. %.

Embodiment 18 is a method of recovering olefins from a C₄ hydrocarbon mixture. The method includes separating the C₄ hydrocarbon mixture in an adsorber section to form: (1) a first olefin stream that contains primarily 1-butene and isobutene collectively, and (2) a first byproduct stream that comprises primarily cis-2-butene, trans-2-butene, n-butane, and isobutane collectively. The method also includes separating the first olefin stream, via molecular sieves, into an isobutene stream containing primarily isobutene and a 1-butene stream containing primarily 1-butene. Embodiment 19 is the method of embodiment 18, wherein the separating of the first olefin stream includes adsorbing isobutene. Embodiment 20 is the method of embodiment 18, wherein the separating of the first olefin stream includes adsorbing 1-butene.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of recovering olefins from a C₄ hydrocarbon mixture, the method comprising:
fractionating the C₄ hydrocarbon mixture in a first separation section to form (1) a first olefin stream comprising primarily 1-butene and isobutene collectively and (2) a first byproduct stream that comprises primarily cis-2-butene and trans-2-butene collectively; and
separating the first olefin stream to form an isobutene stream comprising primarily isobutene and a 1-butene stream comprising primarily 1-butene via a second separation section, wherein the second separation section is adapted to separate hydrocarbon streams by adsorption.

2. The method of claim 1, wherein the first separation section comprises a first distillation column.

3. The method of claim 1, wherein the first olefin stream further comprises isobutane and the first byproduct stream further comprises n-butane.

4. The method of claim 1, wherein the second separation section comprises one or more adsorber unit(s).

5. The method of claim 1, wherein the second separation section separates the first olefin stream into (a) a second olefin stream comprising primarily isobutene and 1-butene collectively and (b) an isobutane stream comprising primarily isobutane.

6. The method of claim 5, further comprising:
separating the second olefin stream into the isobutene stream comprising primarily isobutene and the 1-butene stream comprising primarily 1-butene.

7. The method of claim 6, wherein the separating of the second olefin stream is carried out by a molecular sieve unit.

8. The method claim 1, wherein the second separation section separates the first olefin stream into (a) a second olefin stream comprising primarily isobutene and isobutane collectively and (b) a 1-butene stream comprising primarily 1-butene.

9. The method of claim 8, further comprising:
separating the second olefin stream into the isobutene stream comprising primarily isobutene and an isobutane stream comprising primarily isobutane.

10. The method of claim 9, wherein the separating of the second olefin stream is carried out by a second distillation column.

11. The method of claim 1, wherein the C₄ hydrocarbon mixture is provided to the first separation section from a selection from the list consisting of: a steam cracker, a catalytic cracker, a catalytic dehydrogenation unit, and combinations thereof.

12. The method of claim 1, wherein the first olefin stream comprises (a) 40 wt. % to 80 wt. % of the 1-butene from the C₄ hydrocarbon mixture and (b) 20 wt. % to 50 wt. % of the isobutene from the C₄ hydrocarbon mixture.

13. The method of claim 1, wherein the first byproduct stream comprises 10 wt. % to 30 wt. % of the cis-2-butene from the C₄ hydrocarbon mixture and 40 wt. % to 60 wt. % of the trans-2-butene from the C₄ hydrocarbon mixture.

14. The method of claim 1, wherein the second separation section comprises a selection from the list consisting of: a thermal swing adsorber, a pressure swing adsorber, and combinations thereof.

15. The method of claim 1, wherein the second separation section comprises a plurality of vessels having adsorbent beds and, during operation, at least one of the vessels is in adsorption mode and at least one of the vessels is in regeneration mode.

16. The method of claim 1, wherein rate of recovering isobutene from the C₄ hydrocarbon mixture is at least 60 wt. % and rate of recovering 1-butene from the C₄ hydrocarbon mixture is 60 wt. %.

17. The method of claim 1, wherein the purity of the isobutene is at least 90 wt. % and purity of the 1-butene is at least 90 wt. %.

18. A method of recovering olefins from a C₄ hydrocarbon mixture, the method comprising:
separating the C₄ hydrocarbon mixture in an adsorber section to form (1) a first olefin stream that comprises primarily 1-butene and isobutene collectively and (2) a first byproduct stream that comprises primarily cis-2-butene, trans-2-butene, n-butane, and isobutane collectively; and
separating the first olefin stream, via molecular sieves, into an isobutene stream comprising primarily isobutene and a 1-butene stream comprising primarily 1-butene.

19. The method of claim 18, wherein the separating of the first olefin stream comprises adsorbing isobutene.

20. The method of claim 18, wherein the separating of the first olefin stream comprises adsorbing 1-butene.
